(19) United States
(12) Patent Application Publication
(10) Pub. No.: US 2004/0115286 A1
(43) Pub. Date: Jun. 17, 2004

(54) Cosmetic composition for remedying skin wrinkles comprising bergenia emeiensis extract as active ingredient

(76) Inventors: Kang-Tae Lee, Chunan-shi (KR); Sun-Young Lee, Seoul (KR); Kun-Su Lee, Seoul (KR); Jee-Hean Jeong, Suwon-shi (KR)

Correspondence Address: NIXON & VANDERHYE, PC, 1100 N GLEBE ROAD, 8TH FLOOR, ARLINGTON, VA, 22201-4714, US

(21) Appl. No.: 10/373,826

(22) Filed: Feb. 27, 2003

(30) Foreign Application Priority Data
Dec. 16, 2002 (KR) 2002-80176

(51) Int. Cl. A61K 35/78; A61K 7/06

(52) U.S. Cl. 424/725; 424/74

(57) ABSTRACT

The present invention relates to cosmetic compositions and more particularly to cosmetic compositions for remedying skin wrinklies comprising Bergenia emeiensis extract as an active ingredient. The present invention discloses the novel inhibitory function of Bergenia emeiensis extract on an activity of elastase and collagenase, and provides elastase or collagenase-inhibitory compositions and cosmetic compositions for remedying skin wrinklies comprising the Bergenia emeiensis extract. The compositions of the present invention show significantly enhanced remedying effect of skin wrinkles owing to its efficient inhibition of elastase and collagenase.

### BACKGROUND OF THE INVENTION

1. Field of the Invention

The present invention relates to a cosmetic composition, more particularly, to a cosmetic composition for remedying skin wrinkles comprising Bergenia emeiensis extract as an active ingredient and its applications.

2. Description of the Related Art

The conventional cosmetic compositions for remedying skin wrinkles are aimed to maintain healthy and elastic skin and to prevent and retard skin from aging. Skin is an important part of human body because it is involved in a variety of functions such as protection of body from external stimulations, regulation of body temperature, respiration and excretion. However, these functions become weakened gradually with age and skin aging comes to progress. In particular, if the constitution and content of lipid in lipid layer serving as a skin barrier are changed and thus its functions are sharply declined and the water content of skin is decreased, skin is dried and discoloration, freckles, deposition of pigments and various skin lesions are induced.

Active oxygen and free radicals produced by ultraviolet rays may oxidize the constituents of skin to generate peroxides and in consequence, the structural and functional damages of skin may occur to accelerate skin aging. Especially, if collagen, elastin, proteoglycan, glucosaminoglycan and laminin and the like are oxidized and injured, elasticity of skin may be lost, skin wrinkles may be excessively formed and skin aging may come to occur.

Connective tissue relating to formation of skin wrinkles consists predominantly of fibers such as collagen, reticular and elastic fibers.

Collagen and elastin in dermis tissue are related closely with each other, which can maintain softness and elasticity of skin.

Collagen is a fiber tissue affecting skin softness, a major constituent forming about 70% of dermis tissue. Polypeptide chains of collagen fiber are synthesized with telopeptide at both ends in rough endoplasmic reticulum of fibroblast and secreted into extracellular space in a form of procollagen with triple helix structure. The telopeptide in the secreted procollagens are cleaved by certain proteinase to produce tropocollagens, which in turn are complexed in extracellular matrix to produce collagen fibers. The generation of collagen fibers is sharply dropped with the progress of skin aging, and the modification of collagen fibers by exterior environments may bring about numerous and deep skin wrinkles. It is collagenase that concerns in the cleavage of collagen.

Elastin as well as collagen i. an important fiber that influences skin elasticity, which accounts for 2% of all fiber tissue and has a pivotal role in skin elasticity in spite of its small portion because it forms connective network supporting epidermis. Elastin is cleaved by elastase. Even though elastin is continuously produced by fibroblast until the age of 18-19, it stops being produced after such age. After the stop of elastin production, the skin elasticity is ascribed completely to elastin previously produced, and thus it is very difficult to recover the skin elasticity once the elastin is weakened.

The collagen or elastin is a primary factor that affects the formation of skin wrinkles. That is, skin aging such as formation of skin wrinkles is due to cleavage and reduced formation of collagen and loss of elastin, which are phenomena predominantly occurring in dermis rather than epidermis.

Recently, there have been made many dermatophysiological researches concerning the causes of formation of skin wrinkles and methods for remedying it. In addition, UV sunscreens, antioxidants and cell activity accelerators etc. are used practically in cosmetics and there have been proposed some methods for remedying formation of skin wrinkles.

For example, retinol, widely used for remedying skin wrinkles and preventing pigmentation, is practically used in commercial cosmetics. It has been reported that a cosmetic composition comprising L-ascorbic acid (vitamin C) essential to synthesis of collagen have an effect on blockage of UV rays and prevention of pigmentation (U.S. Pat. Nos. 4,938,969 and 4,772,519 and EP 0533667). It is also known that a cosmetic composition comprising α-hydroxylic acid effective in removing keratin has an effect on promotion of cell activity and skin improvement.

However, most of the active ingredients described above have many shortcomings in terms of safety and stability when used in cosmetics.

Retinol promoting the synthesis of collagen has shortcomings such as oxidization by virtue of light, water, air and heat and resultant declination of stability. Vitamin C shows very weak stability, which is very likely to generate alteration of color and odor in formulations. α-hydroxylic acid shows relatively high skin irritation although it exhibits higher effect in lower acidity.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

Having made intensive investigations on solving these problems to provide active ingredient for remedying skin wrinkles, the present inventors have identified a novel inhibitory function of an extract of Bergenia emeiensis growing in China and other areas against an activity of elastase and collagenase and observed a very excellent effect of cosmetic compositions comprising the Bergenia emeiensis extract on remedy of skin wrinkles with little adverse effect.

Accordingly, it is an object of this invention to provide an inhibitory composition against elastase or collagenase.

It is another object of this invention to provide a cosmetic composition for remedying skin wrinkles.

Other objects and advantages of the present invention will become apparent from the detailed description to follow taken in conjugation with the appended and claims.

### DETAILED DESCRIPTION OF THIS INVENTION

In one aspect of this invention, there is provided a composition for inhibiting elastase or collagenase comprising Bergenia emeiensis extract as an active ingredient.

In another aspect of this invention, there is provided a cosmetic composition for remedying skin wrinkles comprising: (a) Bergenia emeiensis extract as an active ingredient; and (b) a cosmetically acceptable carrier.

In an effort to follow the above need in the art, the present inventors have strived to screen a wide variety of materials to develop an ingredient having excellent effect on remedy of skin wrinkles but not showing lower stability and adverse effects and as a result, have found that an extract of Bergenia emeiensis growing in China and other areas has a very excellent effect on remedy of skin wrinkles and shows little adverse effect.

Bergenia emeiensis belongs to Saxifragaceae, which is 20-30 cm high and big/thick leaves. Its floral color is various from pink to white. Generally, it grows in 1590 meters above the sea level of highlands such as Sichuan, Yunnan and Tibet. Bergenia emeiensis is mainly used with rhizome as folk remedies by Chinese minority race. Its taste is sweet or puckery. It shows functions such as antifebric action, hematostatic action and detoxification to be used in repairing cough and hematemesis and lowering a fever.

The Bergenia emeiensis extract of the present invention shows a function of inhibiting against an activity of elastase and collagenase, so that it can inhibit cleavage of elastin and collagen in dermis and ultimately act on remedying skin wrinkles.

In a preferred embodiment, the Bergenia emeiensis extract may be acquired using various extraction solvents, e.g. (a) water, (b) absolute or hydrous lower alcohol containing 1-4 carbon atoms (methanol, ethanol, propanol, butanol, etc.), (c) mixture of the lower alcohol and water, (d) acetone, (e) ethyl acetate, (f) chloroform, (g) 1,3-butyleneglycol and (h) butyl acetate. More preferable extraction solvent for this invention is the hydrous lower alcohol, and the most preferable solvent is ethanol. Furthermore, it is apparent to one skilled in the art that other conventional solvents may be employed for substantially similar isolating efficiency.

The extract of Bergenia emeiensis according to the present invention can be purified using the known methods in the art. For instance, the isolation and purification may employ gas chromatography (GC), head space gas chromatography (HSGC), liquid chromatography (LC), high performance liquid chromatography (HPLC) and thin layer chromatography (TLC).

The extract of Bergenia emeiensis according to the present invention can be powdered through additional processes such as lyophilization and spray drying.

According to the preferred embodiment of the present invention, the Bergenia emeiensis extract may be obtained by heating Bergenia emeiensis at 40-100° C. for 3-20 hours using an extraction solvent selected from the group consisting of water, anhydrous or hydrous lower alcohol containing 1-4 carbon atoms, acetone, ethyl acetate, butyl acetate, chloroform, 1,3-butyleneglycol and combinations thereof. According to the alternative preferred embodiment of the present invention, the Bergenia emeiensis extract may be obtained by immersing Bergenia emeiensis at 4-50° C. for 1-15 days using an extraction solvent selected from the group consisting of water, anhydrous or hydrous lower alcohol containing 1-4 carbon atoms, acetone, ethyl acetate, butyl acetate, chloroform, 1,3-butyleneglycol and combinations thereof.

The extraction of Bergenia emeiensis extract may be executed with conventional extractors, for example, an extractor equipped with a cooling condenser, which is suitable to prevent active ingredients from evaporating. The drying of the extract may be executed by various methods, for example, rotary vacuous evaporator may be employed.

According to the preferred embodiment of the present invention, the effective amount of Bergenia emeiensis extract in cosmetic composition is 0.0001-10 wt %, and more preferably 0.001-5 wt % based on the total weight of the cosmetic composition.

If the amount of Bergenia emeiensis extract is lower than 0.0001 wt %, the effect of remedying skin wrinkle may be negligible; in the case of exceeding 10 wt %, some adverse effects such as skin irritation and instability in formulation is very likely to occur.

Furthermore, the cosmetic compositions of the present invention may contain auxiliaries as well as carrier in addition to Bergenia emeiensis extract. The non-limiting examples of auxiliaries include preservatives, antioxidants, stabilizers, solubilizers, vitamins, colorants, odor improvers or mixtures of these ingredients.

The cosmetic compositions of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane/butane or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these ingredients.

The formulation of cleansing compositions with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

The following specific examples are intended to be illustrative of the invention and should not be construed as limiting the scope of the invention as defined by appended claims.

### EXAMPLES

Materials

The Bergenia emeiensis employed for extraction was purchased from Chinese Medicine Market.

### Example I

### Preparation of Bergenia emeiensis Extract

### Example I-I

Two hundreds g of washed and dried Bergenia emeiensis was added in 1.2 L of water, heated to 70-90° C. for 5 hours in extractor equipped with a cooling condenser, filtered through 300 mesh filter cloth and stood at 5-10° C. for 7-10 days to ripen and filtered through Whattman No. 5 filter paper. Then, it was dried in a rotary vacuous evaporator at 65° C. 95.5 g of dried powder of Bergenia emeiensis extract was obtained (Extract 1).

### Example I-II

200 g of washed and dried Bergenia emeiensis was immersed in 1.2 L of water at 15-35° C. for 5 days, filtered through 300 mesh filter cloth and filtered again through Whattman No. 5 filter paper and then dried in a rotary vacuous evaporator to concentrate to 2-fold concentration. 0.6 L of 100% ethanol being added, it was stood 5-10° C. for 7-10 days to ripen and filtered through Whattman No. 5 filter paper. Then it was dried in a rotary vacuous evaporator at 65° C. Thus, 98.6 g of dried powder of Bergenia emeiensis extract was obtained (Extract 2).

### Example I-III

200 g of washed and dried Bergenia emeiensis was immersed in 1.2 L of water at 4-40° C. for 5 days, filtered through 300 mesh filter cloth, stood at 5-10° C. for 7-10 days to ripen and filtered through Whattman No. 5 filter paper. Then it was dried in a rotary vacuous evaporator at 65° C. Thus, 90.4 g of dried powder of Bergenia emeiensis extract was obtained (Extract 3).

### Example I-IV

The same processes as the above Example I to III were employed to produce Bergenia emeiensis extracts of the present Example except for the types of extraction solvents used. The dry weight of each extract produced by the processes is shown in Table I, where extracts are represented by Extracts 4 to 21.

TABLE I

| Extract | Extraction solvent | Dry weight (g) |
|---|---|---|
| Extract 4 | 10% ethanol | 98.5 |
| Extract 5 | 20% ethanol | 98.9 |
| Extract 6 | 30% ethanol | 99.2 |
| Extract 7 | 40% ethanol | 96.3 |
| Extract 8 | 50% ethanol | 100.5 |
| Extract 9 | 60% ethanol | 102.3 |
| Extract 10 | 70% ethanol | 110.9 |
| Extract 11 | 80% ethanol | 110.5 |
| Extract 12 | 90% ethanol | 110.2 |
| Extract 13 | 100% ethanol | 110.3 |
| Extract 14 | Methanol | 115.6 |
| Extract 15 | n-propanol | 89.5 |
| Extract 16 | Isopropanol | 88.4 |
| Extract 17 | 2-butanol | 86.3 |
| Extract 18 | Acetone | 85.3 |
| Extract 19 | Chloroform | 84.6 |
| Extract 20 | ethylacetate | 83.6 |
| Extract 21 | butylacetate | 85.3 |

### Example I-V

200 g of washed and dried Bergenia emeiensis was immersed in 1.2 L of 1,4-butyleneglycol for 48 hours, filtered through 300 mesh filter cloth, stood at 5-10° C. for 7-10 days to ripen and filtered through Whattman No. 5 filter paper. The present extract was calculated for reduced dry weight to regulate the final concentration to 1% (w/v) due to difficulty of using rotary vacuous evaporator (Extract 22).

### Example I-VI

200 g of washed and dried Bergenia emeiensis was added in 1.2 L of 10% ethanol, heated for 5 hours in extractor equipped with a cooling condenser, filtered through 300 mesh filter cloth and stood at 5-10° C. for 7-10 days to ripen and filtered through Whattman No. 5 filter paper. Then it was dried in a rotary vacuous evaporator at 65° C. Thus, 110.2 g of dried powder of Bergenia emeiensis extract was obtained (Extract 23).

### Example I-VII

The same processes as the above Example I-VI were employed to produce Bergenia emeiensis extracts of the present Example except for the concentrations of ethanol used. The dry weight of each extract produced by the processes is shown in Table II, where extracts are represented by Extracts 24 to 32.

TABLE II

| Extract | Extraction solvent | Dry weight (g) |
|---|---|---|
| Extract 24 | 20% ethanol | 104.5 |
| Extract 25 | 30% ethanol | 106.9 |
| Extract 26 | 40% ethanol | 108.2 |
| Extract 27 | 50% ethanol | 116.3 |
| Extract 28 | 60% ethanol | 121.5 |
| Extract 29 | 70% ethanol | 125.3 |
| Extract 30 | 80% ethanol | 124.9 |
| Extract 31 | 90% ethanol | 126.5 |
| Extract 32 | 100% ethanol | 128.2 |

### Experimental Example I

### Inhibitory Effect of Bergenia Emeiensis Extracts on Activity of Elastase

The inhibitory effect of the Bergenia emeiensis extracts prepared through the Example I (Extract 1 to 32) on activity of elastase was tested as follows:

20 ul of substrate solution (to 8.8 mM elastase substrate Succ-Ala-Ala-Ala-p-nitroanilide standard solution (Sigma, USA)) was added to 60 ul of buffer (0.267 M Tris regulated to pH 8.0 with 0.267 M HCl), and then 100 ul of the Extract 1 to 32 were added respectively herein and 20 ul of enzyme solution (10 ug/ml of Porcine pancreatic elastase standard (Sigma, USA)) was added to react at 25° C. for 15 minutes. After that, absorbance was measured at 410 nm. The control group contains distilled water instead of Bergenia emeiensis extracts. Inhibitory rate against elastase activity of Bergenia emeiensis extracts was calculated by Equation I.

Inhibitory rate against elastase activity (%)={(absorbance of test group−absorbance of control group)/absorbance of control group}×100 Equation I

### Experimental Example II

### Inhibitory Effect of Bergenia Emeiensis Extracts on Activity of Collagenase

The inhibitory effect of the Bergenia emeiensis extracts prepared through the Example I (Extract 1 to 32) on activity of collagenase was tested as follows:

25 mg of bovine collagen (Sigma, USA) was added to 5 ml of 0.05 M TES and cultured at 37° C. for 15 minutes. Then, collagenase (Sigma, USA) and the Bergenia emeiensis extracts of Extract 1 to 32 were added 100 ug/ml respectively to react at 37° C. for 5 hours (stood with occasional stirring). After that, 0.2 ml of the reaction solution was added to 1 ml of 4% ninhydrin in methyl cellosolve and mixture of 0.2 M sodium citrate and 0.71 mM tin chloride (pH 5.0) following to be heated for 20 minutes. 5 ml of n-propanol were added herein and stood for 15 minutes. After that, absorbance was measured at 600 nm. Inhibitory rate of Bergenia emeiensis extracts against collagenase activity was calculated by Equation II.

Inhibitory rate against collagenase activity (%)={(absorbance of test group−absorbance of control group)/absorbance of control group}×100 Equation II

The results of Experimental Examples I and II are shown in Table III.

TABLE III

| | Inhibitory rate | Inhibitory rate |
|---|---|---|
| | against activity of | against activity of |
| Extract | elastase (%) | collagenase (%) |
| Extract 1 | 60.5 | 67.5 |
| Extract 2 | 61.3 | 65.3 |
| Extract 3 | 60.5 | 66.5 |
| Extract 4 | 61.3 | 69.3 |
| Extract 5 | 62.5 | 67.9 |
| Extract 6 | 63.4 | 69.6 |
| Extract 7 | 66.9 | 67.9 |
| Extract 8 | 68.5 | 68.6 |
| Extract 9 | 75.2 | 76.2 |
| Extract 10 | 77.2 | 79.8 |
| Extract 11 | 76.2 | 75.2 |
| Extract 12 | 77.6 | 76.6 |
| Extract 13 | 78.6 | 75.6 |
| Extract 14 | 78.9 | 76.9 |
| Extract 15 | 73.5 | 66.5 |
| Extract 16 | 72.3 | 65.3 |
| Extract 17 | 66.5 | 59.3 |
| Extract 18 | 75.4 | 67.3 |
| Extract 19 | 73.6 | 68.2 |
| Extract 20 | 73.5 | 69.3 |
| Extract 21 | 72.5 | 67.5 |
| Extract 22 | 78.5 | 68.5 |
| Extract 23 | 75.8 | 67.8 |
| Extract 24 | 75.3 | 64.3 |
| Extract 25 | 74.5 | 66.5 |
| Extract 26 | 73.5 | 68.5 |
| Extract 27 | 75.8 | 67.3 |
| Extract 28 | 76.5 | 66.5 |
| Extract 29 | 78.5 | 68.6 |
| Extract 30 | 76.9 | 67.9 |
| Extract 31 | 75.5 | 66.5 |
| Extract 32 | 76.5 | 67.5 |

As shown in Table III, the inhibitory rates against elastase activity of Bergenia emeiensis extracts according to the present invention were found to be very high running into about 60-80% and that of Extract 14 was the highest, 78.9%. Moreover, inhibitory rates against collagenase activity of Bergenia emeiensis extracts according to the present invention were also found to be very high running into about 60-80% and that of Extract 10 was the highest, 79.8%. These data show the excellent effect of Bergenia emeiensis extracts of the present invention on the inhibition of elastase and collagenase activity.

### Formulation Examples

The formulations of the present invention can be provided in a form of skin softener (skin lotion), astringent lotion, nutrient emulsion (milk lotion), nutrient cream, massage cream, essence, facial pack without limiting the applicable formulations therein.

Formulation I: Skin Lotion (Skin Softener)

One example of skin lotion containing Bergenia emeiensis extract according to the present invention is formulated as Table IV:

TABLE IV

| Ingredients | Amount (wt %) |
|---|---|
| Bergenia emeiensis extract | 5.0 |
| Glycerine | 5.0 |
| 1,3-butylglycol | 3.0 |
| PEG 150 | 1.0 |
| Alantoine | 0.1 |
| DL-pantenol | 0.3 |
| EDTA-2Na | 0.02 |
| Benzophenon-9 | 0.04 |
| Sodium hyaluronate | 5.0 |
| Ethanol | 10.0 |
| Octyldodeces-16 | 0.2 |
| Polysorbate 20 | 0.2 |
| Antiseptic, fragrant, colorant | Small amount |
| DW | To be total |
| Total | 100 |

Formulation II: Astringent Lotion

One example of the astringent lotion containing Bergenia emeiensis extract of the present invention is formulated as Table V:

TABLE V

| Ingredients | Amount (wt %) |
|---|---|
| Bergenia emeiensis extract | 5.0 |
| Glycerine | 2.0 |
| 1,3-butylglycol | 2.0 |
| Alantoine | 0.2 |
| DL-pantenol | 0.2 |
| EDTA-2Na | 0.02 |
| Benzophenon-9 | 0.04 |
| Sodium hyaluronate | 3.0 |
| Ethanol | 15.0 |
| Polysorbate 20 | 0.3 |
| Witchhazel extract | 2.0 |
| Citric acid | Small amount |
| Antiseptic, fragrant, colorant | Small amount |
| DW | To be total |
| Total | 100 |

Formulation III: Nutrient Emulsion (Milk Lotion)

One example of the nutrient emulsion containing Bergenia emeiensis extract of the present invention is formulated as below Table VI:

TABLE VI

| Ingredients | Amount (wt %) |
|---|---|
| Bergenia emeiensis extract | 5.0 |
| Glyceryl stearate SE | 1.5 |
| Stearyl alcohol | 1.5 |
| Lanoline | 1.5 |
| Polysorbate 60 | 1.3 |
| Sorbitan stearate | 0.5 |
| Hydrogenated vegetable oil | 1.0 |
| Mineral oil | 5.0 |
| Squalane | 3.0 |
| Trioctanoine | 2.0 |
| Dimethicon | 0.8 |
| Tocopherol acetate | 0.5 |
| Carboxyvinyl polymer | 0.12 |
| Glycerine | 5.0 |
| 1,3-butylglycol | 3.0 |
| Sodium hyaluronate | 5.0 |
| Tri-ethanolamine | 0.12 |
| Antiseptic, fragrant, colorant | Small amount |
| DW | To be total |
| Total | 100 |

Formulation IV: Nutrient Cream

One example of nutrient cream containing Bergenia emeiensis extract of the present invention is formulated as Table VII:

TABLE VII

| Ingredients | Amount (wt %) |
|---|---|
| Bergenia emeiensis extract | 6.0 |
| Lypophilic glycerol monostearate | 2.0 |
| Cetearyl alcohol | 2.2 |
| Stearic acid | 1.5 |
| Wax | 1.0 |
| Polysorbate 60 | 1.5 |
| Sorbitan stearate | 0.6 |
| Hydrogenated vegetable oil | 1.0 |
| Squalane | 3.0 |
| Mineral oil | 5.0 |
| Trioctanoine | 5.0 |
| Dimethicon | 1.0 |
| Sodium magnesium silicate | 0.1 |
| Glycerine | 5.0 |
| Betaine | 3.0 |
| Tri-ethanolamine | 1.0 |
| Sodium hyaluronate | 4.0 |
| Antiseptic, fragrant, colorant | Small amount |
| DW | To be total |
| Total | 100 |

Formulation V: Message Cream

One example of message cream containing Bergenia emeiensis extract of the present invention is formulated as Table VIII:

TABEL VIII

| Ingredients | Amount (wt %) |
|---|---|
| Bergenia emeiensis extract | 6.0 |
| Lypophilic glycerol monostearate | 1.5 |
| Stearyl alcohol | 1.5 |
| Stearic acid | 1.0 |
| Polysorbate 60 | 1.5 |
| Sorbitan stearate | 0.6 |
| Isostearyl isostearate | 5.0 |
| Squalane | 5.0 |
| Mineral oil | 35.0 |
| Dimethicon | 0.5 |
| Hydroxyethyl cellulose | 0.12 |
| Glycerine | 6.0 |
| Tri-ethanolamine | 0.7 |
| Antiseptic, fragrant, colorant | Small amount |
| DW | To be total |
| Total | 100 |

Formulation VI: Essence

One example of essence containing Bergenia emeiensis extract of the present invention is formulated as Table IX:

TABLE IX

| Ingredients | Amount (wt %) |
|---|---|
| Bergenia emeiensis extract | 5.0 |
| Glycerine | 10.0 |
| Betaine | 5.0 |
| PEG 1500 | 2.0 |
| Alantoine | 0.1 |
| DL-pantenol | 0.3 |
| EDTA-2Na | 0.02 |
| Benzophenon-9 | 0.04 |
| Hydroxyethyl cellulose | 0.1 |
| Sodium hyaluronate | 8.0 |
| Carboxyvinyl polymer | 0.2 |
| Triethanolamine | 0.18 |
| Octyldodecanol | 0.3 |
| Octyldodeces-16 | 0.4 |
| Ethanol | 6.0 |
| Antiseptic, fragrant, colorant | Small amount |
| DW | To be total |
| Total | 100 |

Formulation VII: Facial Pack

One example of facial pack containing Bergenia emeiensis extract of the present invention is formulated as Table X:

TABLE X

| Ingredients | Amount (wt %) |
|---|---|
| Bergenia emeiensis extract | 3.0 |
| Polyvinyl alcohol | 15.0 |
| Cellulose gum | 0.15 |
| Glycerine | 3.0 |
| PEG 1500 | 2.0 |
| Cyclodextrin | 0.15 |
| DL-pantenol | 0.4 |
| Alantoine | 0.1 |
| Monoammonium glycyrrhizinate | 0.3 |
| Nicotineamide | 0.5 |
| Ethanol | 6.0 |
| PEG 40 hydrogenated castor oil | 0.3 |
| Antiseptic, fragrant, colorant | Small amount |
| DW | To be total |
| Total | 100 |

### Experimental Example 3

### Efficacy of the Cosmetic Compositions of the Present Invention on Remedying Skin Wrinkles

The remedying efficacy of skin wrinkles of the cosmetic compositions of the present invention was evaluated through practical applications. The nutrient cream containing 6% Bergenia emeiensis extract described in the Formulation IV was employed and Bergenia emeiensis extract was substituted with the same amount of DW for control in this trial.

At first, 20 women aged from 30 to 40 randomized 2 groups were applied with the nutrient cream of the Formulation IV or its control cream. The application in eyes lasted for 2 months with diurnal twice applications in every morning and night. Remedying efficacy of skin wrinkles was evaluated by observation compared to control groups. The results are summarized in the following Table XI:

TABLE XI

| | | Moderately | | Efficacy |
|---|---|---|---|---|
| | Effective | effective | Ineffective | (%) |
| Formulation IV | 14 | 3 | 3 | 85.0 |
| Control | 3 | 4 | 10 | 35.0 |

As shown in Table XI, the Formulation IV according to the present invention shows significantly enhanced remedying effect of skin wrinkles compared to its control formulation. Furthermore, there was no skin trouble in any testee.

Having described preferred embodiments of the present invention, it is to be understood that variants and modifications thereof falling within the spirit of the invention may become apparent to those skilled in this art, and the scope of this invention is to be determined by appended claims and their equivalents.

What is claimed is:

1. A composition for inhibiting an activity of elstase or collagenase, which comprises Bergenia emeiensis extract as an active ingredient.

2. A cosmetic composition for remedying skin wrinkles comprising: (a) a Bergenia emeiensis extract as an active ingredient; and (b) a cosmetically acceptable carrier.

3. The cosmetic composition according to claim 2, wherein the Bergenia emeiensis extract is present in an amount of 0.0001-10 wt % based on the total weight of the composition.

4. The cosmetic composition according to claims 1 or 2, wherein the Bergenia emeiensis extract is obtained using an extraction solvent selected from the group consisting of water, absolute or hydrous lower alcohol containing 1-4 carbon atoms, acetone, ethyl acetate, butyl acetate, chloroform, 1,3-butyleneglycol and combinations thereof.

5. The cosmetic composition according to claims 1 or 2, wherein the Bergenia emeiensis extract is obtained by heating Bergenia emeiensis at 40-100° C. for 3-20 hours using an extraction solvent selected from the group consisting of water, absolute or hydrous lower alcohol containing 1-4 carbon atoms, acetone, ethyl acetate, chloroform, butyl acetate, 1,3-butyleneglycol and combinations thereof.

6. The cosmetic composition according to claims 1 or 2, wherein the Bergenia emeiensis extract is obtained by immersing Bergenia emeiensis at 4-50° C. for 1-15 days using an extraction solvent selected from the group consisting of water, absolute or hydrous lower alcohol containing 1-4 carbon atoms, acetone, ethyl acetate, butyl acetate, chloroform, 1,3-butyleneglycol and combinations thereof.

7. The cosmetic composition according to claim 2, wherein the composition is in the form of one selected from a solution, a suspension, an emulsion, a paste, an ointment, a gel, a cream, a lotion, a powder, a soap, a surfactant-containing cleanser, an oil, a powder foundation, an emulsion foundation, a wax foundation and a spray.
